(19) United States
(12) Patent Application Publication
(10) Pub. No.: US 2006/0204558 A1
(43) Pub. Date: Sep. 14, 2006

(54) Antimicrobial pet wipes and methods

(76) Inventors: Steven S. Kantner, St. Paul, MN (US); Matthew T. Scholz, Woodbury, MN (US); Danli Wang, Shoreview, MN (US)

Correspondence Address: 3M INNOVATIVE PROPERTIES COMPANY, PO BOX 33427, ST. PAUL, MN, 55133-3427, US

(21) Appl. No.: 11/372,900

(22) Filed: Mar. 10, 2006

Related U.S. Application Data
(60) Provisional application No. 60/660,843, filed on Mar. 10, 2005.

(51) Int. Cl. A61K 31/22; A61K 9/70

(52) U.S. Cl. 424/443; 514/547

(57) ABSTRACT

The present application provides pet wipes with antimicrobial compositions, and methods of using the pet wipes.

### CROSS-REFERENCE TO RELATED APPLICATION

This application claims priority to U.S. Provisional patent Application No. 60/660,843, filed Mar. 10, 2005, which is incorporated herein by reference.

### BACKGROUND

For most pet owners, the grooming and washing of companion animals is necessary to maintain cleanliness both on the pet and in the household as well as maintain a pleasant odor on the animal. Most cleaning of companion animals is done by washing the animal with shampoo or other cleaning compositions. The washing procedure usually involves the use of several implements such as sponges, brushes, shampoo bottles and so forth.

Such companion animals frequently may also develop various skin conditions that affect the appearance of their skin and hair. These disturbances, which may affect skin and hair, can range from pathological conditions of the skin, to fungal infections of the skin, to hyperkeratosis (cornification), to dramatic hair loss, to keratolysis (dissolving or peeling of the keratin from the epidermis), and to other conditions such as pruritus (intense and persistent itching).

Thus, additional compositions are needed in the care of companion animals.

### SUMMARY OF THE INVENTION

The present invention provides pet wipes having antimicrobial activity that are useful for reducing the quantity of microorganisms (including viruses, bacteria, yeast, mold, fungi, mycoplasma, and protozoa), as well as otherwise cleansing and improving the odor of the pet. The pet wipes include an antimicrobial composition that includes an antimicrobial lipid component, optionally a malodor counteractant, optionally an enhancer component, and optionally a surfactant component.

In one embodiment, a wipe for companion animals is provided, comprising a substrate impregnated with an antimicrobial composition, the antimicrobial composition comprising an effective amount of an antimicrobial lipid component comprising a (C7-C14) saturated fatty acid ester of propylene glycol, a (C8-C22) unsaturated fatty acid ester of a propylene glycol, a (C7-C14) saturated fatty ether of a polyhydric alcohol, a (C7-C14) saturated fatty alcohol ester of a (C2-C8) hydroxycarboxylic acid, a (C8-C22) mono- or poly-unsaturated fatty alcohol ester of a (C2-C8) hydroxycarboxylic acid, a (C8-C22) unsaturated fatty ether of a polyhydric alcohol, alkoxylated derivatives thereof, or combinations thereof, wherein the alkoxylated derivative has less than 5 moles of alkoxide per mole of polyhydric alcohol or hydroxycarboxylic acid; a malodor counteractant; and an aqueous phase.

In another embodiment, a wipe for companion animals is provided comprising a substrate impregnated with an antimicrobial composition, the antimicrobial composition comprising an effective amount of an antimicrobial lipid component comprising a (C7-C14) saturated fatty acid ester of propylene glycol, a (C8-C22) unsaturated fatty acid ester of propylene glycol; a malodor counteractant; and an aqueous phase.

In one embodiment, a wipe for companion animals is provided, comprising a substrate impregnated with an antimicrobial composition, the antimicrobial composition comprising an effective amount of an antimicrobial lipid component comprising a (C7-C14) saturated fatty acid ester of propylene glycol, a (C8-C22) unsaturated fatty acid ester of a propylene glycol, a (C7-C14) saturated fatty ether of a polyhydric alcohol, a (C7-C14) saturated fatty alcohol ester of a (C2-C8) hydroxycarboxylic acid, a (C8-C22) mono- or poly-unsaturated fatty alcohol ester of a (C2-C8) hydroxycarboxylic acid, a (C8-C22) unsaturated fatty ether of a polyhydric alcohol, alkoxylated derivatives thereof, or combinations thereof, wherein the alkoxylated derivative has less than 5 moles of alkoxide per mole of polyhydric alcohol or hydroxycarboxylic acid; and an aqueous phase, wherein the impregnated antimicrobial composition has a viscosity less than 500 cps.

In another embodiment, a wipe for companion animals is provided comprising a substrate impregnated with an antimicrobial composition comprising a (C7-C14) saturated fatty alcohol monoester of a (C2-C8) hydroxycarboxylic acid, a (C8-C22) mono- or poly-unsaturated fatty alcohol monoester of a (C2-C8) hydroxycarboxylic acid, alkoxylated derivatives thereof, or combinations thereof, wherein the alkoxylated derivative has less than 5 moles of alkoxide per mole of hydroxycarboxylic acid.

For certain embodiments, the antimicrobial lipid component includes propylene glycol monolaurate, propylene glycol monocaprate, propylene glycol monocaprylate, or combinations thereof.

For certain embodiments, the antimicrobial lipid component is present in an amount of at least 0.1 wt-%.

For certain embodiments, the antimicrobial lipid component includes no greater than 40 wt-%, based on the total weight of the antimicrobial lipid component, of a di- or tri-ester, a di- or tri-ether, alkoxylated derivative thereof, or combinations thereof.

For certain embodiments, antimicrobial compositions used in the pet wipes of the present invention can further include an effective amount of an enhancer component distinct from the antimicrobial lipid component. For preferred embodiments, the enhancer component can include a chelating agent.

For certain embodiments, the enhancer component includes an alpha-hydroxy acid, a beta-hydroxy acid, a chelating agent, a (C1-C4) alkyl carboxylic acid, a (C6-C12) aryl carboxylic acid, a (C7-C12) aralkyl carboxylic acid, a (C7-C12) alkaryl carboxylic acid, a phenolic compound, a (C1-C10) alkyl alcohol, an ether glycol, or combinations thereof. For certain embodiments, the total concentration of the enhancer component relative to the total concentration of lipid component is within a range of 10:1 to 1:300, on a weight basis.

For certain embodiments, antimicrobial compositions used in the pet wipes of the present invention can further include an effective amount of a surfactant component distinct from the antimicrobial lipid component. For certain embodiments, the surfactant component can include a nonionic surfactant, an anionic surfactant, a cationic surfactant, or mixtures thereof. In preferred embodiment, the surfactant is a nonionic surfactant such as alkyl polyglucoside. For certain embodiments, the total concentration of the surfactant component to the total concentration of antimicrobial lipid component is within a range of 1:1 to 1:100, on a weight basis.

Definitions

As used herein, the term “companion animals” refers to animals commonly domesticated by people and used as companionship pets. This could include, for example, dogs and cats, but otherwise may also include more exotic pets.

“Effective amount” means the amount of the antimicrobial lipid component and the enhancer component (when present in a composition) and/or the surfactant component (when present in a composition), that as a whole, provides an antimicrobial (including, for example, antiviral, antibacterial, or antifungal) activity that reduces, prevents, or eliminates one or more species of microbes such that an acceptable level of the microbe results. Typically, this is a level low enough not to cause clinical symptoms, and is desirably a non-detectable level. It should be understood that in the compositions of the present invention, the concentrations or amounts of the components, when considered separately, may not kill to an acceptable level, or may not kill as broad a spectrum of undesired microorganisms, or may not kill as fast; however, when used together such components provide an enhanced (preferably synergistic) antimicrobial activity as compared to the same components used alone under the same conditions.

“Enhancer” means a component that enhances the effectiveness of the antimicrobial lipid component such that when the composition less the antimicrobial lipid component and the composition less the enhancer component are used separately, they do not provide the same level of antimicrobial activity as the composition as a whole. For example, an enhancer component in the absence of the antimicrobial lipid component may not provide any appreciable antimicrobial activity. The enhancing effect can be with respect to the level of kill, the speed of kill, and/or the spectrum of microorganisms killed, and may not be effective for all microorganisms. In fact, an enhanced level of kill is most often seen in Gram negative bacteria such as Escherichia coli. An enhancer may be a synergist such that when combined with the remainder of the composition, the composition as a whole displays an activity that is greater than the sum of the activity of the composition less the enhancer component and the composition less the antimicrobial lipid component.

“Microorganism” or “microbe” or “microorganism” refers to bacteria, yeast, mold, fungi, protozoa, mycoplasma, as well as viruses (including lipid enveloped RNA and DNA viruses).

“Antiseptic” means a chemical agent that kills pathogenic and non-pathogenic microorganisms. Preferred antiseptics exhibit at least a 4 log reduction of both P. aeruginosa and S. aureus in 60 minutes from an initial inoculum of 1-3×10⁷ cfu/ml when tested in Mueller Hinton broth at 35° C. at a concentration of 0.25 wt-% in a Rate of Kill assay using an appropriate neutralizer as described in “The Antimicrobial Activity in vitro of chlorhexidine, a mixture of isothiazolinones (Kathon CG) and cetyl trimethyl ammonium bromide (CTAB),” G. Nicoletti et al., Journal of Hospital Infection, 23, 87-111 (1993). Antiseptics generally interfere with the cellular metabolism and/or the cell envelope. Antiseptics are sometimes referred to as disinfectants, especially when used to treat hard surfaces.

“Antimicrobial lipid” means an antiseptic having at least one alkyl or alkylene group having at least 6 carbon atoms, more preferably at least 7 carbon atoms, and more preferably at least 8 carbon atoms. The antimicrobial lipids preferably have a hydrophile/lipophile balance (HLB) of at most 6.2, more preferably at most 5.8, and even more preferably at most 5.5. The antimicrobial lipids preferably have an HLB of at least 3, preferably at least 3.2, and even more preferably at least 3.4.

“Fatty” as used herein refers to a straight or branched chain alkyl or alkylene moiety having at least 6 carbon atoms, unless otherwise specified.

The term “comprises” and variations thereof do not have a limiting meaning where these terms appear in the description and claims.

As used herein, “a,” “an,” “the,” “at least one,” and “one or more” are used interchangeably. The term “and/or” means one or all of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### DETAILED DESCRIPTION OF ILLUSTRATIVE EMBODIMENTS

The present invention provides pet wipes that include antimicrobial compositions that include an antimicrobial lipid component. The compositions of such wipes have antimicrobial activity and are useful for reducing microorganisms (including viruses, bacteria, yeast, mold, fungi, mycoplasma, and protozoa), and additionally useful for otherwise cleansing and improving the odor of the pet.

The wipe described herein provides a very mild “leave-on” formulation for cleansing the skin or hair of a mammal while providing softness to the substrate and conditioning to the skin or hair. The lipophilic antimicrobial lipid component provides emolliency that improves the condition of skin and hair and also can impart long lasting antimicrobial benefit, particularly when coupled with an enhancer component such as an acid or chelating agent.

An optional water-soluble antimicrobial agent, such as benzethonium chloride or benzalkonium chloride can further enhance effectiveness. Other antimicrobial agents distinct from the antimicrobial lipid component include for example, peroxides, (C6-C14) alkyl carboxylic acids and alkyl ester carboxylic acids, antimicrobial natural oils, as described in Applicants' Assignee's Copending U.S. patent application Ser. No. 10/936,133, filed on Sep. 7, 2004; halogenated phenols, diphenyl ethers, bisphenols (including but not limited to p-chloro m-xylenol (PCMX) and triclosan), and halogenated carbanilides described in Applicants' Assignee's Copending U.S. patent application Ser. No 10/936,171, filed on Sep. 7, 2004; digluconate, diacetate, dimethosulfate, and dilactate salts; polymeric quaternary ammonium compounds such as polyhexamethylenebiguanide; silver and various silver complexes; other small molecule quaternary ammonium compounds; di-long chain alkyl (C8-C18) quaternary ammonium compounds; cetylpyridinium halides and their derivatives; and octenidine described in Applicants' Assignee's Copending U.S. patent application Ser. No. 10/936,135, filed on Sep. 7, 2004; and compatible combinations thereof.

Preferred compositions include a malodor counteractant; such as those molecules designed to specifically bind or react with (and thereby neutralize) odor causing organic amines, sulfides, thiols, and acids. The malodor counteractant provides rapid deodorization of the treated skin or hair. Malodor counteractants include but are not limited to Ordenone™ by Belle-Aire Fragrance, Odor Trap™ by U.S. Flavor and Fragrance, Flexisorb OD-100 by Innovative Chemical Technology Inc., Meelium™ by Prentiss Inc., Tego Sorb™ Conc. 50 by Goldschmidt Chemical Corp., undecylenic acid and derivatives, uncomplexed cyclodextrin, zeolites and the like. The concentration of the malodor counteractant is typically from 0% to about 5% or less, and preferably about at least 0.5% to about at most 2% of the composition. The malodor counteractants listed above can be used alone or in combination.

Preferred compositions also include humectants. A humectant is a polar hygroscopic material that increases hydration by drawing water from the environment to help retain water in the skin's upper layers. Suitable humectants include, but are not limited to, glycol, urea, and glycerol. Other moisturizers that may be suitable include those described in U.S. patent application Ser. No. 11/077,864, filed Mar. 10, 2005.

This pre-saturated wet wipe provides a convenient and economical method of cleansing the coat of a fur-bearing animal to rapidly reduce malodor, while at the same time preventing its re-occurrence by suppressing the growth of odor causing bacterial. The wipe is particularly suited for regular use on companion animals.

When a thinner, but still durable, substrate is used, a soft, non-abrasive wet wipe for cleansing a mammal's ears is provided. The detergency of the non-ionic surfactant and the lipophilicity of the antimicrobial lipid component serve to soften and loosen accumulations of earwax, while the optional skin care actives can sooth irritation and aid healing in cases where the outer ear is inflamed.

Antimicrobial Lipid Component

The antimicrobial lipid component is that component of the composition that provides at least part of the antimicrobial activity. That is, the antimicrobial lipid component has at least some antimicrobial activity for at least one microorganism. It is generally considered the main active component of the compositions of the present invention.

The antimicrobial lipids preferably have a hydrophile/lipophile balance (HLB) of at most 6.2, more preferably at most 5.8, and even more preferably at most 5.5. The antimicrobial lipids preferably have an HLB of at least 3, preferably at least 3.2, and even more preferably at least 3.4.

Preferred antimicrobial lipids are uncharged and have an alkyl or alkenyl hydrocarbon chain containing at least 6 carbon atoms.

In certain embodiments, the antimicrobial lipid component preferably includes one or more fatty acid esters of a polyhydric alcohol, fatty ethers of a polyhydric alcohol, or alkoxylated derivatives thereof (of either or both of the ester and ether), or combinations thereof. More specifically and preferably, the antimicrobial component is selected from the group consisting of a (C7-C14) saturated fatty acid ester of a polyhydric alcohol (preferably, a (C7-C12) saturated fatty acid ester of a polyhydric alcohol, and more preferably, a (C8-C12) saturated fatty acid ester of a polyhydric alcohol); a (C8-C22) unsaturated fatty acid ester of a polyhydric alcohol (preferably, a (C12-C22) unsaturated fatty acid ester of a polyhydric alcohol); a (C7-C14) saturated fatty ether of a polyhydric alcohol (preferably, a (C7-C12) saturated fatty ether of a polyhydric alcohol, and more preferably, a (C8-C12) saturated fatty ether of a polyhydric alcohol); a (C8-C22) unsaturated fatty ether of a polyhydric alcohol (preferably, a (C12-C22) unsaturated fatty ether of a polyhydric alcohol); a (C7-C14) saturated fatty alcohol ester of a (C2-C8) hydroxycarboxylic acid (preferably, a (C7-C12) saturated fatty alcohol ester of a (C2-C8) hydroxycarboxylic acid, more preferably, a (C8-C12) saturated fatty alcohol ester of a (C2-C8) hydroxycarboxylic acid); a (C8-C22) mono- or poly-unsaturated fatty alcohol ester of a (C2-C8) hydroxycarboxylic acid; an alkoxylated derivative thereof; and combinations thereof. Preferably, the esters and ethers are monoesters and monoethers, unless they are esters and ethers of sucrose in which case they can be monoesters, diesters, monoethers, or diethers. Various combinations of monoesters, diesters, monoethers, and diethers can be used in a composition of the present invention.

Exemplary fatty acid monoesters include, but are not limited to, propylene glycol monoesters of lauric, caprylic, and capric acid, as well as lauric, caprylic, and capric acid monoesters of sucrose. Other fatty acid monoesters include glycerin and propylene glycol monoesters of oleic (18:1), linoleic (18:2), linolenic (18:3), and arachonic (20:4) unsaturated (including polyunsaturated) fatty acids.

The compositions of the present invention include one or more fatty acid esters, fatty ethers, fatty alcohol esters, alkoxylated fatty acid esters, alkoxylated fatty ethers, alkoxylated fatty alcohol esters, and combinations thereof, at a suitable level to produce the desired result. Such compositions preferably include a total amount of such material of at least 0.01 percent by weight (wt-%), more preferably at least 0.1 wt-%, even more preferably at least 0.25 wt-%, even more preferably at least 0.5 wt-%, and even more preferably at least 1 wt-%, based on the total weight of the “ready to use” or “as used” composition. In a preferred embodiment, they are present in a total amount of no greater than 20 wt-%, more preferably no greater than 15 wt-%, even more preferably no greater than 10 wt-%, and even more preferably no greater than 5 wt-%, based on the “ready to use” or “as used” composition. Certain compositions may be higher in concentration if they are intended to be diluted prior to use.

Preferred compositions of the present invention that include one or more fatty acid monoesters, fatty monoethers, fatty alcohol monoesters, or alkoxylated derivatives thereof can also include a small amount of a di- or tri-fatty acid ester (i.e., a fatty acid di- or tri-ester), a di- or tri-fatty ether (i.e., a fatty di- or tri-ether), or alkoxylated derivative thereof. Preferably, such components are present in an amount of no more than 50 wt-%, more preferably no more than 40 wt-%, even more preferably no more than 25 wt-%, even more preferably no more than 15 wt-%, even more preferably no more than 10 wt-%, even more preferably no more than 7 wt-%, even more preferably no more than 6 wt-%, and even more preferably no more than 5 wt-%, based on the total weight of the antimicrobial lipid component. For example, for monoesters, monoethers, or alkoxylated derivatives of glycerin, preferably there is no more than 15 wt-%, more preferably no more than 10 wt-%, even more preferably no more than 7 wt-%, even more preferably no more than 6 wt-%, and even more preferably no more than 5 wt-% of a diester, diether, triester, triether, or alkoxylated derivatives thereof present, based on the total weight of the antimicrobial lipid components present in the composition. However, as will be explained in greater detail below, higher concentrations of di- and tri-esters may be tolerated in the raw material if the formulation initially includes free glycerin because of transesterification reactions.

Although in some situations it is desirable to avoid di- or tri-esters as a component of the starting materials, it is possible to use relatively pure tri-esters in the preparation of certain compositions of the present invention (for example, as a hydrophobic component) and have effective antimicrobial activity.

Enhancer Component

Compositions of the present invention preferably include an enhancer (preferably a synergist) to enhance the antimicrobial activity especially against Gram negative bacteria, such as E. coli and Pseudomonas sp. The chosen enhancer preferably affects the cell envelope of the bacteria. While not bound by theory, it is presently believed that the enhancer functions by allowing the antimicrobial lipid to more easily enter the cell cytoplasm and/or by facilitating disruption of the cell envelope. The enhancer component may include an alpha-hydroxy acid, a beta-hydroxy acid, other carboxylic acids, a phenolic compound (such as certain antioxidants and parabens), a monohydroxy alcohol, a chelating agent, or a glycol ether (i.e., ether glycol). Various combinations of enhancers can be used if desired.

The alpha-hydroxy acid, beta-hydroxy acid, and other carboxylic acid enhancers are preferably present in their protonated, free acid form. It is not necessary for all of the acidic enhancers to be present in the free acid form; however, the preferred concentrations listed below refer to the amount present in the free acid form. Additional non-alpha hydroxy acid, beta-hydroxy acid or other carboxylic acid enhancers may be added in order to acidify the formulation or buffer it at a pH to maintain antimicrobial activity. Furthermore, the chelator enhancers that include carboxylic acid groups are preferably present with at least one, and more preferably at least two, carboxylic acid groups in their free acid form. The concentrations given below assume this to be the case.

One or more enhancers may be used in the compositions of the present invention at a suitable level to produce the desired result. In a preferred embodiment, they are present in a total amount greater than 0.01 wt-%, more preferably in an amount greater than 0.1 wt-%, even more preferably in an amount greater than 0.2 wt-%, even more preferably in an amount greater than 0.25 wt-%, and most preferably in an amount greater than 0.4 wt-% based on the total weight of the ready to use composition. In a preferred embodiment, they are present in a total amount of no greater than 20 wt-%, based on the total weight of the ready to use composition. Such concentrations typically apply to alpha-hydroxy acids, beta-hydroxy acids, other carboxylic acids, chelating agents, phenolics, ether glycols, and (C5-C10) monohydroxy alcohols. Generally, higher concentrations are needed for (C1-C4) monohydroxy alcohols.

The alpha-hydroxy acid, beta-hydroxy acid, and other carboxylic acid enhancers, as well as chelators that include carboxylic acid groups, are preferably present in a concentration of no greater than 100 milliMoles per 100 grams of formulated composition. In most embodiments, alpha-hydroxy acid, beta-hydroxy acid, and other carboxylic acid enhancers, as well as chelators that include carboxylic acid groups, are preferably present in a concentration of no greater than 75 milliMoles per 100 grams, more preferably no greater than 50 milliMoles per 100 grams, and most preferably no greater than 25 milliMoles per 100 grams of formulated composition.

The total concentration of the enhancer component relative to the total concentration of the antimicrobial lipid component is preferably within a range of 10:1 to 1:300, and more preferably 5:1 to 1:10, on a weight basis.

An additional consideration when using an enhancer is the solubility and physical stability in the compositions. Many of the enhancers discussed herein are insoluble in hydrophobic components.

Alternatively, the enhancer may be present in excess of the solubility limit provided that the composition is physically stable. This may be achieved by utilizing a sufficiently viscous composition that stratification (e.g., settling or creaming) of the antimicrobial lipid does not appreciably occur.

In certain preferred embodiments, the chelating agents useful in the compositions of the present invention include those selected from the group consisting of ethylenediaminetetraacetic acid (EDTA) and salts thereof, succinic acid, and mixtures thereof. Preferably, either the free acid or the mono- or di-salt form of EDTA is used.

One or more chelating agents may be used in the compositions of the present invention at a suitable level to produce the desired result. In a preferred embodiment, they are present in a total amount of at least 0.01 wt-%, more preferably at least 0.05 wt-%, even more preferably at least 0.1 wt-%, and even more preferably at least 0.5 wt-%, based on the weight of the ready to use composition. In a preferred embodiment, they are present in a total amount of no greater than 10 wt-%, more preferably no greater than 5 wt-%, and even more preferably no greater than 1 wt-%, based on the weight of the ready to use composition.

The ratio of the total concentration of chelating agents (other than alpha- or beta-hydroxy acids) to the total concentration of the antimicrobial lipid component is preferably within a range of 10:1 to 1:100, and more preferably 1:1 to 1:10, on a weight basis.

Surfactants

Compositions of the present invention can optionally include one or more surfactants. In some embodiments, the presence of a surfactant may be used to emulsify the composition and to help wet the surface and/or to aid in contacting the microorganisms. As used herein the term “surfactant” means an amphiphile (a molecule possessing both polar and nonpolar regions which are covalently bound) capable of reducing the surface tension of water and/or the interfacial tension between water and an immiscible liquid. The term is meant to include soaps, detergents, emulsifiers, surface active agents, and the like. The surfactant can be cationic, anionic, nonionic, or amphoteric. This includes a wide variety of conventional surfactants. Combinations of various surfactants can be used if desired.

Certain ethoxylated surfactants can reduce or eliminate the antimicrobial efficacy of the antimicrobial lipid component. The exact mechanism of this is not known and not all ethoxylated surfactants display this negative effect. For example, poloxamer (polyethylene oxide/polypropylene oxide) surfactants have been shown to be compatible with the antimicrobial lipid component, but ethoxylated sorbitan fatty acid esters such as those sold under the trade name TWEEN by ICI have not been compatible. It should be noted that these are broad generalizations and the activity could be formulation dependent.

It should be noted that certain antimicrobial lipids are amphiphiles and may be surface active. For example, certain antimicrobial alkyl monoglycerides described herein are surface active. For certain embodiments of the invention, the antimicrobial lipid component is considered distinct from a “surfactant” component.

Preferred nonionic surfactants are those that have an HLB (i.e., hydrophile to lipophile balance) of at least 4 and more preferably at least 8. Even more preferred surfactants have an HLB of at least 12. Most preferred surfactants have an HLB of at least 15; however, lower HLB surfactants are still useful in compositions described herein.

In certain preferred embodiments, the surfactants useful in the compositions of the present invention are selected from the group consisting of sulfonates, sulfates, phosphonates, phosphates, poloxamer (polyethylene oxide/polypropylene oxide block copolymers), alkyl glucosides, alkyl polyglycosides, cationic surfactants, and mixtures thereof. In certain more preferred embodiments, the surfactants useful in the compositions of the present invention are selected from the group consisting of poloxamer, alkyl glucosides, alkyl polyglycosides, and mixtures thereof.

One or more surfactants may be used in the compositions of the present invention at a suitable level to produce the desired result. In a preferred embodiment, they are present in a total amount of at least 0.1 wt-%, more preferably at least 0.5 wt-%, and even more preferably at least 1.0 wt-%, based on the total weight of the ready to use composition.

Surfactants may be present in a total amount of no greater than 10 wt-%, more preferably no greater than 5 wt-%, even more preferably no greater than 3 wt-%, and even more preferably no greater than 2 wt-%, based on the total weight of the ready to use composition. The ratio of the total concentration of surfactant to the total concentration of the antimicrobial lipid component is preferably within a range of 5:1 to 1:100, more preferably 3:1 to 1:10, and most preferably 2:1 to 1:3, on a weight basis.

Optional Additives

Compositions described herein may additionally employ adjunct components useful in physically formulating various dosage forms of the present invention, such as excipients, dyes, skin conditioning agents, hair conditioning agents, hair styling agents, shine imparting agents, perfumes, lubricants, thickening agents, stabilizers, skin penetration agents, preservatives, or antioxidants.

It will be appreciated by the skilled artisan that the levels or ranges selected for the required or optional components described herein will depend upon whether one is formulating a composition for direct use, or a concentrate for dilution prior to use, as well as the specific component selected, the ultimate end-use of the composition, and other factors well known to the skilled artisan.

It will also be appreciated that additional antiseptics (i.e., disinfectants) may be included and are contemplated. These include, for example, “azole” antifungal agents including clortrimazole, miconazole, econazole, ketoconazole, and salts thereof; and the like.

Formulations and Methods of Preparation

Many of the compositions described herein have exceptional broad spectrum antimicrobial activity and thus are generally not terminally sterilized but if necessary may be sterilized by a variety of industry standard techniques. For example, it may be preferred to sterilize the compositions in their final packaged form using electron beam. It may also be possible to sterilize the sample by gamma radiation or heat. Other forms of sterilization may be acceptable. It may also be suitable to include preservatives in the formulation to prevent growth of certain organisms. Suitable preservatives include industry standard compounds such as parabens (methyl, ethyl, propyl, isopropyl, isobutyl, etc.); 2 bromo-2 nitro-1,3 diol; 5 bromo-5-nitro-1,3 dioxane; chlorbutanol; diazolidinyl urea; iodopropylnyl butylcarbamate; phenoxyethanol; halogenated cresols; methylchloroisothiazolinone; and the like, as well as combinations of these compounds.

The compositions described herein preferably adhere well to animal hair and tissues in order to deliver the composition to the intended site over a prolonged period even in the presence of moisture. The component in the greatest amount (i.e., the vehicle) in the formulations of the invention may be any conventional vehicle commonly used for topical treatment of animal skin. The formulations are typically selected from one of the following three types: (1) anhydrous or nearly anhydrous formulations with a hydrophobic vehicle (i.e., one or more hydrophobic compounds present in the greatest amount); (2) anhydrous or nearly anhydrous formulations with a hydrophilic vehicle (i.e., one or more hydrophilic compounds present in the greatest amount); and (3) aqueous-based formulations.

Aqueous-based formulations are presently preferred. Such formulations preferably contain greater than 85% water, more preferably greater than 90% water, and even more preferably greater than 95% water. The antimicrobial lipid component is preferably emulsified or solubilized into this aqueous based formulation by the surfactant component, which also provides wetting, cleansing and soil suspending properties in use. Other hydrophobic components, such as emollients or certain shine enhancing additives can be emulsified or solubilized by the surfactant component as well. The aqueous based formulation may also include water soluble materials such as humectants as well as water swellable materials such as crosslinked polymers used as thickening agents which may aid in stabilizing the formulation. Even when thickening agents are use, preferred compositions have a viscosity less than 500 centipoise, preferable less than 200 centipoise, more preferably less than 100 centipoise, still more preferably less than 50 centipoise, even more preferably less than 20 centipoise, and most preferably less than 10 centipoise. Such lower viscosity formulations wick quickly into a stack of dry precut wipes, enabling the high speed production of wet wipe products of this invention.

Delivery Methods and Devices

Typically, the compositions are delivered to the mammalian tissue by toweling or wiping the pet wipe in a manner that allows at least a portion of the composition to spread and perhaps penetrate into the tissue, (as opposed to through the tissue into the blood stream). The wipe can include a foam, knit, woven, or nonwoven material.

The antimicrobial cleansing composition is coated impregnated at the desired weight onto one or both sides of an absorbent sheet (hereinafter sometimes referred to as “substrate”) which may be formed from any woven or nonwoven fiber, fiber mixture or foam of sufficient wet strength and absorbency to hold an effective amount of the composition. It is preferred from the standpoint of antimicrobial effectiveness and mildness to employ substrates with a high absorbent capacity (e.g., from about 5 to about 20 grams/gram, preferably from about 9 to about 20 grams/gram). The absorbent capacity of a substrate is the ability of the substrate, while supported horizontally, to hold liquid.

In particular, woven or nonwoven fabrics derived from “oriented” or carded fibrous webs composed of textile-length fibers, the major proportion of which are oriented predominantly in one direction are suitable for use herein. These fabrics can be in the form of, for example, wipes or towelettes, including baby wipes and the like.

Thermocarded nonwoven cloths (whether or not resin-containing) are made of polyesters, polyamides, or other thermoplastic fibers which can be spun bonded, i.e., the fibers are spun out onto a flat surface and bonded (melted) together by heat or chemical reactions.

The nonwoven cloth substrates used in the invention herein are generally adhesively bonded fibers or filamentous products having a web or carded fiber structure (when the fiber strength is suitable to allow carding) or comprising fibrous mats in which the fibers or filaments are distributed haphazardly or in random array (i.e., an array of fibers in a carded web where partial orientation of the fibers is frequently present, as well as a completely haphazard distributional orientation), or substantially aligned. The fibers or filaments can be natural (e.g., wool, silk, jute, hemp, cotton, linen, sisal, or ramie) or synthetic (e.g., rayon, cellulose ester, polyvinyl derivatives, polyolefins, polyamides, or polyesters) as have been described hereinabove. These nonwoven materials are generally described in Riedel “Nonwoven Bonding Methods and Materials”, Nonwoven World, (1987).

Objects and advantages of this invention are further illustrated by the following examples, but the particular materials and amounts thereof recited in these examples, as well as other conditions and details, should not be construed to unduly limit this invention. Unless otherwise indicated, all parts and percentages are on a weight basis, all water is deionized water, and all molecular weights are weight average molecular weight.

### EXAMPLES

### Example 1

An aqueous emulsion was prepared by dissolving glycerin and alkyl polyglycoside in the water in a stainless steel kettle at room temperature. The propylene glycol monolaurate was then emulsified into this solution with moderate agitation, mixing for 1 hour. To the resulting hazy solution, the remaining ingredients were added, and agitation was continued for 45 minutes more before draining. It was used as a saturant for a Z-folded flat stack of twelve 8″×10″ non-woven cloths consisting of needlepunched 50/50 rayon/polyester of 110 gsm basis weight. Excess saturant was squeezed out to give a 240 weight % added solution to the cloth.

| Ingredient | CAS # | % |
|---|---|---|
| Water | 7732-18-5 | 95-98%% |
| Glycerin | 56-81-5 | 1-3% |
| Propylene Glycol | 27194-74-7 | 0.5-1.5% |
| Monolaurate | | |
| alkyl polyglycoside | 110615-47-9 | 0.5-1.5% |
| aloe barbadensis leaf juice | 85507-69-3 | <0.1% |
| tocopheryl acetate | 58-95-7 | <0.1% |
| simethicone | 8050-81-5 | <0.1% |
| Polysorbate 20 | 9005-64-5 | <0.2% |
| Benzethonium Chloride | 121-54-0 | 0.1-0.2% |
| Ordenone ™ | NA | <0.3% |
| Disodium EDTA | 139-33-3 | 0.05% |
| diazolidinyl urea | 78491-02-8 | <0.5% |
| phenoxyethanol | 122-99-6 | <0.8% |
| methylparaben | 99-76-3 | <0.3% |
| ethylparaben | 120-47-8 | <0.1% |
| butyl paraben | 94-26-8 | <0.1% |
| propylparaben | 95-13-3 | <0.1% |
| isobutylparaben | 4247-02-3 | <0.1% |
| fragrance | NA | <0.3% |

### Example 2

The solution and process of Example 1 was used to prepare a Z-folded flat stack of twenty 4″×6″ non-woven cloths consisting of needlepunched 70/30 rayon/polyester of 40 gsm basis weight (Ahlstrom 17002).

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

What is claimed is:

1. A wipe for companion animals comprising a substrate impregnated with an antimicrobial composition comprising:
an effective amount of an antimicrobial lipid component comprising a (C7-C14) saturated fatty acid ester of propylene glycol, a (C8-C22) unsaturated fatty acid ester of a propylene glycol, a (C7-C14) saturated fatty ether of a polyhydric alcohol, a (C7-C14) saturated fatty alcohol ester of a (C2-C8) hydroxycarboxylic acid, a (C8-C22) mono- or poly-unsaturated fatty alcohol ester of a (C2-C8) hydroxycarboxylic acid, a (C8-C22) unsaturated fatty ether of a polyhydric alcohol, alkoxylated derivatives thereof, or combinations thereof, wherein the alkoxylated derivative has less than 5 moles of alkoxide per mole of polyhydric alcohol or hydroxycarboxylic acid;
a malodor counteractant; and
an aqueous phase.

2. The pet wipe of claim 1 wherein the antimicrobial composition further comprises an effective amount of an enhancer component.

3. The pet wipe of claim 2 wherein the enhancer component comprises an alpha-hydroxy acid, a beta-hydroxy acid, a chelating agent, a (C1-C4) alkyl carboxylic acid, a (C6-C12) aryl carboxylic acid, a (C7-C12) aralkyl carboxylic acid, a (C7-C12) alkaryl carboxylic acid, a phenolic compound, a (C1-C10) alkyl alcohol, an ether glycol, or combinations thereof.

4. The pet wipe of claim 3 wherein the enhancer component comprises a chelating agent.

5. The pet wipe of claim 2 wherein the total concentration of the enhancer component relative to the total concentration of lipid component is within a range of 10:1 to 1:300, on a weight basis.

6. The pet wipe of claim 1 wherein the composition further comprises an effective amount of a surfactant component distinct from the antimicrobial lipid component.

7. The pet wipe of claim 6 wherein the surfactant component comprises a sulfonate surfactant, a sulfate surfactant, a phosphonate surfactant, a phosphate surfactant, a poloxamer surfactant, an alkyl glucoside surfactant, an alkyl polyglycoside surfactant, a cationic surfactant, or mixtures thereof.

8. The pet wipe of claim 7 wherein the surfactant component is an alkyl glucoside surfactant, an alkyl polyglycoside surfactant, or mixtures thereof.

9. The pet wipe of claim 6 wherein the total concentration of the surfactant component to the total concentration of antimicrobial lipid component is within a range of 5:1 to 1:100, on a weight basis.

10. The pet wipe of claim 1 wherein the antimicrobial lipid component comprises glycerol monolaurate, glycerol monocaprate, glycerol monocaprylate, propylene glycol monolaurate, propylene glycol monocaprate, propylene glycol monocaprylate, or combinations thereof.

11. The pet wipe of claim 1 wherein the antimicrobial lipid component is present in an amount of at least 0.1 wt-%.

12. The pet wipe of claim 1 wherein the antimicrobial lipid component includes no greater than 40 wt-%, based on the total weight of the antimicrobial lipid component, of a di- or tri-ester, a di- or tri-ether, alkoxylated derivative thereof, or combinations thereof.

13. The pet wipe of claim 1 wherein the antimicrobial composition has a viscosity of less than 500 centipoise.

14. The pet wipe of claim 1, further comprising an antimicrobial agent separate from the antimicrobial lipid component.

15. The pet wipe of claim 1, further comprising a humectant.

16. The pet wipe of claim 1, wherein the antimicrobial lipid component comprises a (C7-C14) saturated fatty acid ester of propylene glycol, a (C8-C22) unsaturated fatty acid ester of propylene glycol, and combinations thereof.

17. A wipe for companion animals comprising a substrate impregnated with an antimicrobial composition comprising:
an effective amount of an antimicrobial lipid component comprising a (C7-C14) saturated fatty acid ester of propylene glycol, a (C8-C22) unsaturated fatty acid ester of a propylene glycol, a (C7-C14) saturated fatty ether of a polyhydric alcohol, a (C7-C14) saturated fatty alcohol ester of a (C2-C8) hydroxycarboxylic acid, a (C8-C22) mono- or poly-unsaturated fatty alcohol ester of a (C2-C8) hydroxycarboxylic acid, a (C8-C22) unsaturated fatty ether of a polyhydric alcohol, alkoxylated derivatives thereof, or combinations thereof, wherein the alkoxylated derivative has less than 5 moles of alkoxide per mole of polyhydric alcohol or hydroxycarboxylic acid; and
an aqueous phase,
wherein the impregnated antimicrobial composition has a viscosity less than 500 cps.

18. The pet wipe of claim 17 wherein the antimicrobial composition further comprises an effective amount of an enhancer component.

19. The pet wipe of claim 18 wherein the enhancer component comprises an alpha-hydroxy acid, a beta-hydroxy acid, a chelating agent, a (C1-C4) alkyl carboxylic acid, a (C6-C12) aryl carboxylic acid, a (C7-C12) aralkyl carboxylic acid, a (C7-C12) alkaryl carboxylic acid, a phenolic compound, a (C1-C10) alkyl alcohol, an ether glycol, or combinations thereof.

20. The pet wipe of claim 18 wherein the total concentration of the enhancer component relative to the total concentration of lipid component is within a range of 10:1 to 1:300, on a weight basis.

21. The pet wipe of claim 17 wherein the composition further comprises an effective amount of a surfactant component distinct from the antimicrobial lipid component.

22. The pet wipe of claim 21 wherein the surfactant component comprises a sulfonate surfactant, a sulfate surfactant, a phosphonate surfactant, a phosphate surfactant, a poloxamer surfactant, an alkyl glucoside surfactant, an alkyl polyglycoside surfactant, a cationic surfactant, or mixtures thereof.

23. The pet wipe of claim 21 wherein the total concentration of the surfactant component to the total concentration of antimicrobial lipid component is within a range of 5:1 to 1:100, on a weight basis.

24. The pet wipe of claim 17 wherein the antimicrobial lipid component comprises glycerol monolaurate, glycerol monocaprate, glycerol monocaprylate, propylene glycol monolaurate, propylene glycol monocaprate, propylene glycol monocaprylate, or combinations thereof.

25. The pet wipe of claim 17 wherein the antimicrobial lipid component includes no greater than 40 wt-%, based on the total weight of the antimicrobial lipid component, of a di- or tri-ester, a di- or tri-ether, alkoxylated derivative thereof, or combinations thereof.

26. The pet wipe of claim 17, further comprising a humectant.

27. A wipe for companion animals comprising a substrate impregnated with an antimicrobial composition comprising a (C7-C14) saturated fatty alcohol monoester of a (C2-C8) hydroxycarboxylic acid, a (C8-C22) mono- or poly-unsaturated fatty alcohol monoester of a (C2-C8) hydroxycarboxylic acid, alkoxylated derivatives thereof, or combinations thereof, wherein the alkoxylated derivative has less than 5 moles of alkoxide per mole of hydroxycarboxylic acid.

28. The pet wipe of claim 27 wherein the antimicrobial composition further comprises an effective amount of an enhancer component.

29. The pet wipe of claim 27 wherein the composition further comprises an effective amount of a surfactant component distinct from the antimicrobial lipid component.

30. The pet wipe of claim 27, further comprising a humectant.
